(19) United States
(12) Patent Application Publication
(10) Pub. No.: US 2020/0282134 A1
(43) Pub. Date: Sep. 10, 2020

(54) IV SPIKE FOR USE WITH NON-ISO COMPLIANT IV CONTAINER

(71) Applicant: CareFusion 303, Inc., San Diego, CA (US)

(72) Inventor: Benjamin Yang Teck TAN, Singapore (SG)

(21) Appl. No.: 16/292,195

(22) Filed: Mar. 4, 2019

(51) Int. Cl. A61M 5/162; A61J 1/20; A61M 5/168

(52) U.S. Cl. CPC A61M 5/162; A61M 5/1411; A61M 5/168; A61J 1/2006

(57) ABSTRACT

An intravenous (IV) spike for administering a medicinal fluid from a container includes an elongate body having an upper portion and a lower portion, and a plurality of screw threads disposed along and protruding radially outward from an outer surface of the lower portion of the elongate body. The elongate body is configured to be coupled to a drip chamber. The outer surface is configured to engage an internal surface of an outlet port of the container in a coupled configuration. In the coupled configuration edges of the screw threads grip and engage the internal surface of the outlet port of the container to create a seal between the lower portion of the elongate body and the outlet port of the container and to retain the body in the outlet port.

### TECHNICAL FIELD

The present disclosure generally relates to IV set components, and more particularly to IV spikes for puncturing and sealing membranes of non-ISO compliant IV fluid bags and bottles.

### BACKGROUND

One of the most widely used methods of medical therapy is the intravenous (IV) infusion of liquid medicaments and/or nutrients into the bloodstream of a patient. A familiar apparatus that is used in many IV infusion applications is an IV container, such as an IV bag or bottle, which contains the liquid to be infused into the patient.

When the IV container is a bag or bottle, a rigid, hollow, sharpened IV spike is pushed into the bag to establish a pathway for fluid communication through which the liquid can flow out of the bag. The spike is usually inserted into the bag through a sealed membrane, commonly referred to as a port. In turn, the spike is connected to or formed integrally with an inlet port of a small, elongated, transparent hollow container familiarly referred to as a “drip chamber,” with the fluid pathway of the spike in fluid communication with an interior of the drip chamber.

IV containers in the form of bags or bottles as discussed above may generally be classified as Intentional Organization for Standardization (ISO) compliant or non-ISO compliant. Where non-ISO compliant containers are pierced (“spiked”) using existing IV spikes, there is a higher tendency or risk for the non-ISO compliant containers to leak upon spiking.

### SUMMARY

In accordance with some embodiments, an intravenous (IV) spike for administering a medicinal fluid from a container includes an elongate body having an upper portion and a lower portion, the elongate body configured to be coupled to a drip chamber; a spike head disposed at the upper portion of the elongate body, the spike head having a puncture tip and a puncture base having a fluid inlet at an upper end thereof, wherein a fluid channel extends from the fluid inlet, through the elongate body, and into the drip chamber; a plurality of screw threads disposed along and protruding radially outward from an outer surface of the lower portion of the elongate body, wherein: the outer surface is configured to engage an internal surface of an outlet port of the container in a coupled configuration; and in the coupled configuration edges of the screw threads grip and engage the internal surface of the outlet port of the container to create a seal between the lower portion of the elongate body and the outlet port of the container and to retain the body in the outlet port.

In accordance with some embodiments, an intravenous (IV) drip system may include an IV container containing a fluid and including a fluid outlet port having an internal surface, the internal surface being configured without a sealing member; a spike having an elongate body including an upper portion, a lower portion, and a plurality of screw threads disposed along and protruding radially outward from an outer surface of the elongate body, wherein: the outer surface of the lower portion is configured to engage the internal surface of the fluid outlet port of the IV container in a coupled configuration; and in the coupled configuration, the screw threads penetrate at least partially into the internal surface of the outlet port of the IV container to create a seal between the spike and the outlet port of the container and to retain the spike in the outlet port.

In accordance with some embodiments, a method of manufacturing a spike for an intravenous (IV) drip system includes providing an elongate body with a spike tip at an upper portion thereof and a base at a lower portion thereof, forming a plurality of screw threads protruding radially outward from an outer surface of the base of the elongate body; and positioning a sealing member between a pair of adjacent threads of the plurality of screw threads.

Aspects and features of the models and methods disclosed herein can be provided, excluded, or modified based on the teachings and disclosure herein.

### BRIEF DESCRIPTION OF THE DRAWINGS

The following figures are included to illustrate certain aspects of the embodiments, and should not be viewed as exclusive embodiments. The subject matter disclosed is capable of considerable modifications, alterations, combinations, and equivalents in form and function, as will occur to those skilled in the art and having the benefit of this disclosure.

FIG. 1A depicts a perspective view of a conventional IV spike.

FIG. 1B is a cross-sectional view of an IV set that includes a non-ISO compliant IV container spiked by the conventional IV spike of FIG. 1A.

FIG. 1C is an enlarged partial cross-sectional view of the IV set that includes the non-ISO compliant IV container spiked by the conventional IV spike of FIG. 1B.

FIG. 2A depicts a perspective view of an IV spike, in accordance with some embodiments of the present disclosure.

FIG. 2B depicts a cross-sectional view of the IV spike of FIG. 2A, in accordance with some embodiments of the present disclosure.

FIG. 2C is a cross-sectional view of an IV set that includes a non-ISO compliant IV container spiked by IV spike of FIG. 2A, in accordance with some embodiments of the present disclosure.

FIG. 2D is an enlarged partial cross-sectional view of the IV set that includes the non-ISO compliant IV container spiked by the IV spike of FIG. 2A.

FIGS. 3A and 3B depict a method of spiking and sealing the non-ISO compliant IV container with the IV spike of FIG. 2A, in accordance with some embodiments of the present disclosure.

### DETAILED DESCRIPTION

Various embodiments of the present disclosure are directed to providing IV spikes for puncturing (“spiking”) membranes of non-ISO compliant IV fluid bags and bottles, where the IV spikes have improved retention features for retaining the IV spike in the IV container.

Various embodiments of the present disclosure are additionally directed to providing IV spikes for spiking membranes of non-ISO compliant IV fluid bags and bottles, where the IV spikes have improved sealing capabilities so as to prevent fluid from inadvertently leaking between external surfaces of the IV spikes and internal surfaces of the IV fluid bags and bottles.

Embodiments disclosed herein are directed to providing an IV spike with improved sealing and retention capabilities as compared with existing IV spikes. In accordance with some embodiments, the IV spike 120 may generally include a body having an upper portion and a lower portion or base. The lower portion or base of the IV spike may provide sealing and retention mechanisms thereon for sealingly and securely engaging the IV spike in the outlet port during spiking of the non-ISO compliant IV bag or bottle. In particular, as depicted, the lower portion may include a plurality of screw threads disposed along and protruding radially outward from an outer surface of the lower portion. In some embodiments, the screw threads may act as the retention mechanism to anchor and retain the body of the IV spike in the non-ISO compliant IV bag or bottle.

In a coupled or “spiked” configuration where the IV spike is inserted or spiked into the outlet port of the non-ISO compliant IV container, edges of the screw threads may at least partially penetrate the internal surface of the outlet port to grip and engage the internal surface of the outlet port 116 of the non-ISO compliant IV container. Penetration of the screw threads into the internal surface 112 of the outlet port 116 creates a first seal between the IV spike and the outlet port of the non-ISO compliant IV container. Advantageously, the seal created prevents fluid from inadvertently leaking between the IV spike and the outlet port of the non-ISO compliant IV container 102 upon, during, or after spiking.

In some embodiments, the screw threads may also act as a retaining mechanism for improving the ability of the non-ISO compliant IV container to retain the IV spike inserted or “spiked” therein. The screw threads may thus prevent the IV spike from separating (or otherwise dislodging) from the non-ISO compliant IV container. As the screw threads 136 rotate and penetrate or otherwise “bite” or “dig” into the internal surface of the outlet port, material of the internal surface of the outlet port is compressed between the screw threads 136, which further increases the resistance of the non-ISO compliant IV container to pull-out and to shear loads. Advantageously, the screw threads may thus serve as a structure that increases friction between the IV spike and the outlet port such that the IV spike may not be easily dislodged from the non-ISO compliant IV container, without departing from the scope of the disclosure.

In accordance with various embodiments of the present disclosure, the IV spike may be configured with a sealing member disposed on the lower portion of the IV spike body, and interposed between a pair of adjacent screw threads. In the coupled configuration the sealing member plugs and forms a second seal between the internal surface of the outlet port and the outer surface of the lower portion of the IV spike body. As the sealing member 135 is advanced into the outlet port 116 of the non-ISO compliant IV container 102, the sealing member 135 seals the path created by the penetration of the screw threads 136. The IV spike 120 thus remedies the deficiencies of the existing IV spike 20 by incorporating the sealing member thereon as a secondary seal to further prevent fluid from inadvertently leaking between the IV spike 120 and the outlet port 116 of the non-ISO compliant IV container 102 in the coupled configuration.

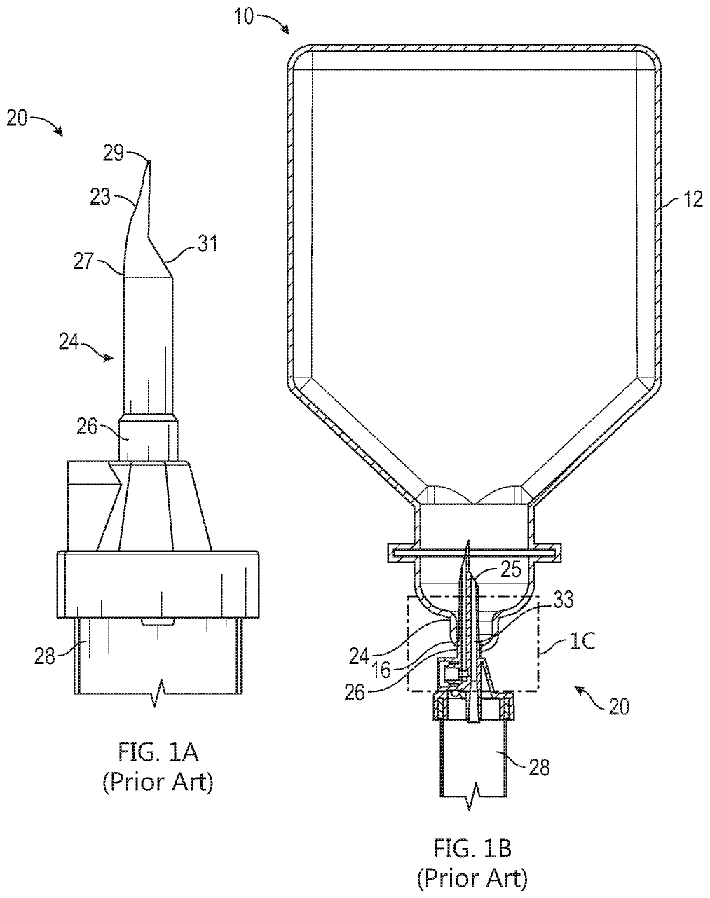

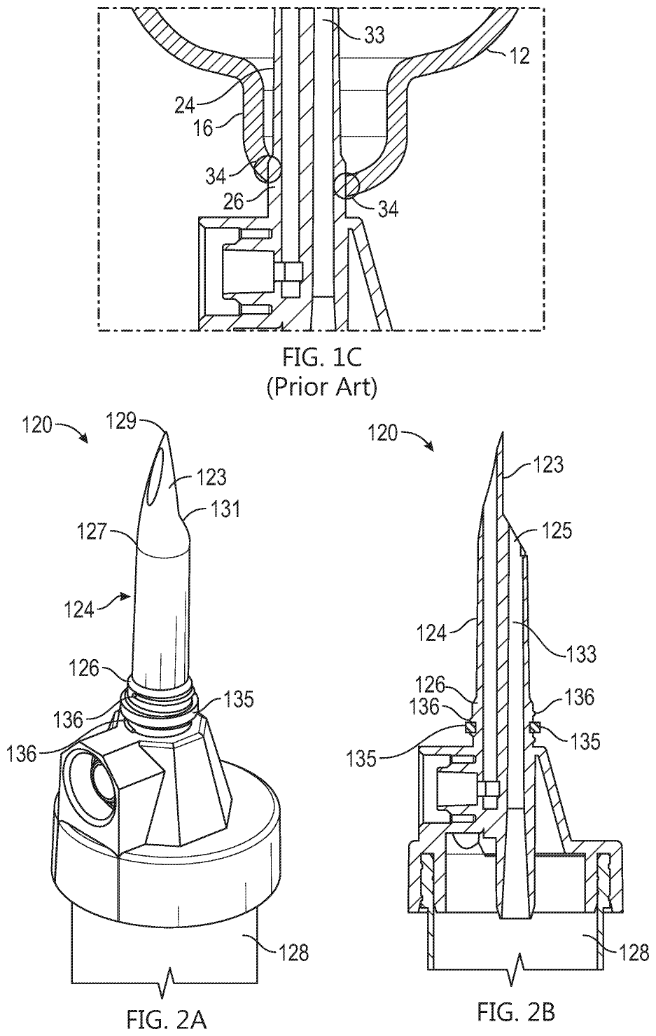

FIG. 1A depicts a perspective view of a conventional IV spike 20. FIG. 1B is a cross-sectional view of an IV set 10 that includes an IV container 12 spiked by the conventional IV spike 20 of FIG. 1A. FIG. 1C is an enlarged partial cross-sectional view of the IV set that includes the IV container 12 spiked by the conventional IV spike of FIG. 1B. The IV container 12 may be a non-ISO compliant IV fluid bag or bottle. As described herein a non-ISO compliant IV bag or bottle is one having an outlet port, e.g., outlet port 16, without any form of sealing structure or sealing member thereon. For example, in contrast to non-ISO compliant IV bags or bottles, ISO compliant IV bags or bottles generally include some form of sealing element on the outlet port in the form of a rubber bung, plug, or stopper for sealingly coupling the outlet port and the conventional IV spike 20 during spiking. The sealing elements incorporated into the ISO compliant IV bags thus are capable of minimizing fluid leaks between the internal surface of the outlet port and the outer surface of the body of the conventional IV spike 20.

As depicted, the conventional IV spike 20 may generally include a body 24 having an upper portion 27 and a lower portion 26. The conventional IV spike 20 may be configured to be coupled to a drip chamber 28. A spike 23 having a puncture tip 29 and a puncture base 31 may be disposed at the upper portion 27 of the body 24. The puncture base 31 may have a fluid inlet 25 at an upper end thereof. As illustrated, a fluid channel 33 may extend from the puncture base 31, through the body 24 and into the drip chamber 28. The lower portion 26 may serve as a base of the IV spike 20 and may have an outer diameter greater than an outer diameter of the upper portion 27 the elongate body 24. In a coupled configuration, as illustrated in FIG. 1B, in which the non-ISO compliant IV container 12 is pierced or spiked by the IV spike 20, fluid leaks may occur at the contact points 34 of the base 24 and the internal surface of the outlet port 16 as a result of the lack of some form of a sealing element interposed therebetween.

Therefore, it would be advantageous to have an IV spike capable of sufficiently sealing a non-ISO compliant IV bag or bottle so as to prevent leakage of fluid from the non-ISO compliant IV bag or bottle upon, during, and/or after spiking. It is further advantageous to have an IV spike having improved retention features over conventional or currently existing IV spikes for retaining the IV spike in the non-ISO compliant IV bag or bottle. The various embodiments of the present disclosure are directed to providing an IV spike having the aforementioned features that are lacking in the conventional or currently existing IV spikes.

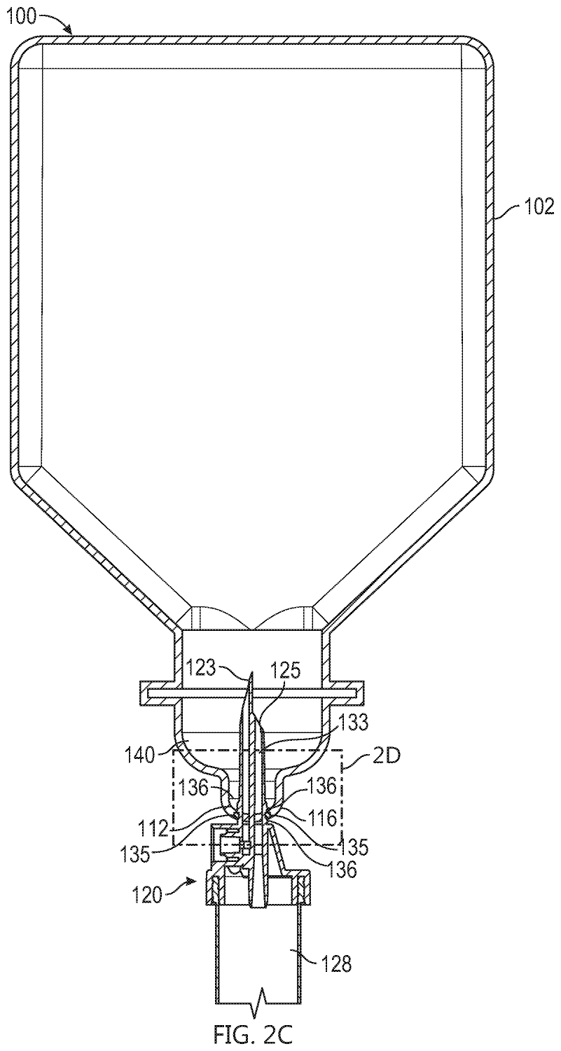

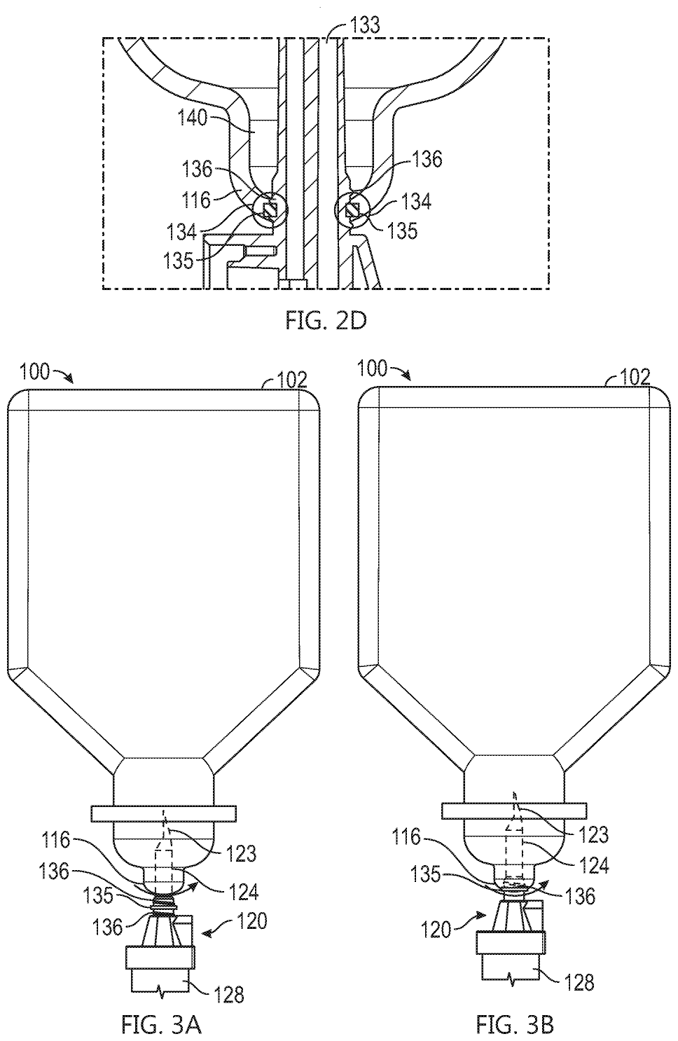

FIG. 2A depicts a perspective view of an IV spike 120, in accordance with some embodiments of the present disclosure. FIG. 2B depicts a cross-sectional view of the IV spike 120 of FIG. 2A, in accordance with some embodiments of the present disclosure. FIG. 2C is a cross-sectional view of an IV set that includes an IV container 102 spiked by IV spike 120 of FIG. 2A, in accordance with some embodiments of the present disclosure. FIG. 2D is an enlarged partial cross-sectional view of the IV set 100 that includes the IV container 102 spiked by the IV spike 120 of FIG. 2A.

As illustrated in FIGS. 2C and 2D, the IV container 102 may be a non-ISO compliant IV fluid bag or bottle. As described above, a non-ISO compliant IV bag or bottle is discussed herein as an IV bag or bottle having an outlet port, e.g., outlet port 116, without any form of sealing structure or sealing member thereon. For example, in contrast to non-ISO compliant IV bags or bottles, ISO compliant IV bags or bottles generally include some form of sealing element on the outlet port in the form of a rubber bung, plug, or stopper for sealingly coupling the outlet port and the IV spike during spiking. The sealing elements incorporated into the outlet ports of existing ISO compliant IV bags thus are capable of minimizing fluid leaks between the internal surface of the outlet port and the outer surface of the body of the existing IV spikes. In contrast, due to a lack of some form of sealing member or element being incorporated onto the outlet port or nozzle of the non-ISO compliant IV bags or bottles, fluid may leak out of the outlet port during, or after spiking.

Various embodiments of the present disclosure are directed to providing an IV spike 120 having improved sealing and retention capabilities as compared with existing IV spikes, e.g., IV spike 20. Referring back to FIGS. 2A and 2B, the IV spike 120 may generally include a body 124 having an upper portion 127 and a lower portion or base 126. The IV spike 120 may be configured to be coupled to a drip chamber 128. A spike head 123 having a puncture tip 129 and a puncture base 131 may be disposed at the upper portion 127 of the body 124. According to various aspects of the present disclosure, the lower portion 126 may also be referred to herein as a base 126 of the IV spike body 124 and may have an outer diameter greater than an outer diameter of the upper portion 27 the elongate body 24. The aforementioned configuration may be advantageous in that a tight interference fit may be formed between the base 126 of the IV spike 120 and the internal surface 112 of the outlet port 116. The tight interference fit may further aid in retention of the IV spike 120 in the non-ISO compliant IV bag or bottle 102.

According to various aspects of the present disclosure, the lower portion or base 126 of the IV spike 120 may provide sealing and retention mechanisms thereon for sealingly and securely engaging the IV spike 20 in the outlet port 116 during spiking of the non-ISO compliant IV bag or bottle 102. In particular, as depicted, the lower portion 126 may include a plurality of screw threads 136 disposed along and protruding radially outward from an outer surface of the lower portion 126. In some embodiments, the screw threads 136 may anchor the body 124 of the IV spike 20 into the non-ISO compliant IV bag or bottle 102.

In accordance with various embodiments, the shape and configuration of the plurality of screw threads 136 is not limited to any particular configuration. The screw threads 136 may have an apex having a sharpness sufficient to engage and “bite into the outlet port of the non-ISO compliant IV bag or bottle 102. In some embodiments, a pitch of the plurality of screw threads 136 can be varied. For example, the plurality of screw threads 136 may be formed with a shallow thread pitch which provides many turns of threads 136 around the outer surface of the lower portion 126 of the body 124 with adjacent threads 136 spaced closely together. Alternatively, the plurality of screw threads 136 may be formed with a steep thread pitch which provides few turns of threads 136 with adjacent threads 136 spaced further apart than the configuration having the steep pitch. In some embodiments, the height of the screw threads 136 from the outer surface of the lower portion 126 of the IV spike body 124 to the apex of the thread may extend and protrude radially outward a sufficient height to dig or otherwise cut into the internal surface 112 of the outlet port 116. Accordingly, an outer diameter of each of the screw threads may be larger than an internal diameter of the outlet port.

In some embodiments, the plurality of screw threads 136 may be formed at least partially along the lower portion 126 of the body 124. In other embodiments, the plurality of screw threads 136 may be formed completely along the lower portion 126 of the body 124. As depicted in FIG. 2A, the plurality of screw threads 136 may be formed in a helical configuration along the lower portion 126 of the body 124. However, the various embodiments of the present disclosure are not limited to the aforementioned configuration. In some embodiments, the screw threads 136 are formed in a series of radial bands along the lower portion 126 of the body 124. In some embodiments, the screw threads 136 may be spaced apart from each other at regular intervals, e.g., equally spaced apart. In other embodiments however, the screw threads 136 may be spaced apart from each other at irregular intervals, e.g., spaced apart at varying intervals.

As depicted in FIG. 2A, the plurality of screw threads 136 may form a helical spiral around the lower portion 126 of the body 124. The number of threads and thread pitch are parameters that may be varied depending on the material of the outlet port 116 of the IV container 102 in which the IV spike 120 is to be inserted. For example, if the screw threads 136 are to anchor the IV spike 120 in the outlet port 116, then the thread pitch and other screw parameters should be selected so that the screw can withstand large shearing and axial loads. If the screw threads 136 are to be used for other purposes, then other factors will dictate the selection of screw design parameters.

In accordance with various embodiments, the outer surface of the IV spike 120 is configured to engage an internal surface 112 of an outlet port 116 of the container in a coupled configuration. As discussed further herein, a coupled configuration refers to a configuration in which the IV spike 120 is inserted or spiked into the outlet port 116 of the non-ISO compliant IV container 102. As depicted in FIG. 2D, in the coupled configuration, edges of at least one of the screw threads 136 at least partially penetrate the internal surface 112 of the outlet port 116 to grip and engage the internal surface 112 of the outlet port 116 of the non-ISO compliant IV container 102. As such, the screw threads 136 may be formed with an undercut so as to sufficiently “bite into,” “penetrate,” or otherwise engage the internal surface 112 of the outlet port 116. Penetration of the screw threads 136 into the internal surface 112 of the outlet port 116 creates a seal between the IV spike 120 and the outlet port 116 of the non-ISO compliant IV container 102. Advantageously, the seal created prevents fluid 140 from inadvertently leaking between the IV spike 120 and the outlet port 116 of the non-ISO compliant IV container 102.

In some embodiments, the screw threads 136 may also act as a retaining mechanism for improving the ability of the non-ISO compliant IV container 102 to retain the IV spike 120 inserted therein in the coupled configuration. The screw threads 136 may thus prevent the IV spike 120 from separating (or otherwise dislodging) from the non-ISO compliant IV container 102. As the screw threads 136 rotate and penetrate or otherwise “bite” or “dig” into the internal surface 112 of the outlet port 116, material of the internal surface 112 of the outlet port 116 is compressed between the screw threads 136. The pinching of the material of the internal surface 112 of the outlet port 116 between the screw threads further increases the resistance of the non-ISO compliant IV container 102 to pull-out and to shear loads. Advantageously, the screw threads 136 may thus be structure that increases friction between the IV spike 120 and the outlet port 116 such that the IV spike 120 may not be easily dislodged from the non-ISO compliant IV container 102, without departing from the scope of the disclosure.

In some embodiments, the screw threads 136 may be structured as a ramp that has a slight undercut configured to penetrate, “bite into,” or otherwise “dig into” the internal surface 112 of the outlet port 116 as the IV spike 120 is inserted or advanced into the non-ISO compliant IV container 102. When the IV spike 120 is pulled to be withdrawn from the non-ISO compliant IV container 102, the threads will secure the IV spike 120 within the non-ISO compliant IV container 102. In some embodiments, the top of the ramp, or the undercut portion of the screw threads 136, will dig into or grip the internal surface 112 of the outlet port 116 when the IV spike 120 is attempted to be withdrawn from within the non-ISO compliant IV container 102. Advantageously as a result, the tensile force required to remove or otherwise dislodge the IV spike 120 from the non-ISO compliant IV container 102 is increased and thus the IV spike 120 is better secured in the non-ISO compliant IV container 102.

It should be noted that the locations of the screw threads on the outer surface of the IV spike 120 in the Figures are merely examples, and the location of the screw threads 136 may be changed, without departing from the scope of the disclosure. Further, although the Figures indicate a plurality of screw threads 136, the plurality of screw threads 136 may be replaced, for example with ledges and/or barbed features, the number of which may not be limited and may be increased or decreased, without departing from the scope of the disclosure. For example, multiple ledges may be disposed at regular intervals along the outer surface of the lower portion 126 of the IV spike body 124. However, in other embodiments, the ledges may be disposed at irregular intervals along the outer surface of the lower portion 126 of the IV spike body, 124. Similarly, multiple barbed features may be disposed at regular intervals along the outer surface of the lower portion 126 of the IV spike body, 124. However, in other embodiments, the barbed features may be disposed at irregular intervals. The circumferential extent of the barbed features radially outward from the spike body 124 may be increased or decreased as required by application or design, and without departing from the scope of the disclosure.

The IV spike 120 of the various embodiments described herein thus yields further advantages over the currently existing IV spike 20 when coupled to a non-ISO compliant IV container, e.g., containers 12, and 102. In particular, when the currently existing IV spike 20 is in the coupled configuration, as illustrated in FIGS. 1B and 1C, where the non-ISO compliant IV container 12 is pierced or spiked by the IV spike 20. fluid leaks may occur at the contact points 34 of the base 24 and the internal surface 21 of the outlet port 16. As previously discussed, the leaks may occur as a result of lack of a sealing element or mechanism interposed between the base 24 and the internal surface 21 of the outlet port 16. The IV spike 120 of the various embodiments described herein remedies the deficiencies of the existing IV spike 20 by incorporating the screw threads 136 thereon as a sealing surface which at least partially penetrates or “digs into” the internal surface 112 of the outlet port 116 when the IV spike 120 is inserted or “spiked” into the outlet port 116 of the non-ISO compliant IV container 102. The screw threads 136 may cut a helical path into the internal of the outlet port 16 as the IV spike 120 rotates into the outlet port 116. Further advantageously, because the screw threads 136 rotate and penetrate or otherwise “bite” or “dig” into the internal surface 112 of the outlet port 116, the screw threads 136 may thus prevent the IV spike 120 from separating (or otherwise dislodging) from the non-ISO compliant IV container 102. The screw threads 136 advantageously keep the IV spike 120 from being axially pulled out of the non-ISO compliant IV container 102.

As described above, a non-ISO compliant IV bag or bottle is discussed herein as an IV bag or bottle having an outlet port, e.g., outlet port 116, without any form of sealing structure or sealing member thereon. Thus, in accordance with various embodiments of the present disclosure, the internal surface 112 of the outlet port 116 of the IV container 102 (e.g., a non-ISO compliant IV bag or bottle) may be devoid of a sealing member. For example, in contrast to some ISO compliant IV bags and bottles having a sealing member disposed on the outlet port, the IV container 102 may lack or otherwise exclude a sealing member on the internal surface 112 thereof. As such, when the IV container 12 is spiked with the currently existing IV spike 20, fluid leaks may occur at the contact points 34 of the base 24 and the internal surface 21 of the outlet port 16.

The IV spike 120 of the various embodiments described herein remedies the deficiencies of the existing IV spike 20 by incorporating a sealing member 135 on the body 124 of the IV spike 120. In particular, as illustrated in FIGS. 2A-2D, the sealing member 135 may be disposed on the lower portion 126 of the spike and interposed between a pair of adjacent screw threads 136. As the sealing member 135 is advanced into the outlet port 116 of the non-ISO compliant IV container 102, the sealing member 135 seals the path created by the penetration of the screw threads 136. Thus the sealing member 135 acts as a secondary seal to further prevent fluid 140 from inadvertently leaking between the IV spike 120 and the outlet port 116 of the non-ISO compliant IV container 102 in the coupled configuration.

In some embodiments, the sealing member comprises a material selected from the group consisting of rubber, polytetrafluoroethylene (PTFE), silicone, and any combination thereof. Additionally, the sealing member may be made of synthetic and natural elastomers and elastomeric compounds of any chemical type that can be processed by at least one of, or a combination of injection molding, compression molding, transfer molding, casting and extrusion techniques. Examples of such existing elastomers can be found in FDA 21 CFR 177.2600, however the various embodiments of the present disclosure are not limited to this particular regulatory standard.

According to various aspects of the present disclosure, the puncture base 131 may have a fluid inlet 125 at an upper end thereof. As illustrated, a fluid channel 133 may extend from the puncture base 131, through the body 124 and into the drip chamber 128. As such, fluid 140 from the IV container 102 may enter the IV spike 120 at the fluid inlet 125 and flow through the fluid channel 133 into the drip chamber 128.

FIGS. 3A and 3B depict a method of spiking and sealing the IV container with the IV spike of FIG. 2A, in accordance with some embodiments of the present disclosure. As illustrated in FIG. 3A, the IV spike 120 is inserted into the outlet port 116 of the non-ISO compliant IV bag or bottle 102 with a tight interference fit between the lower portion 126 of the IV spike body 124 and the internal surface 112 of the outlet port 116. The IV spike 120 is then rotated and pushed further up into the outlet port 116 of the non-ISO compliant IV bag or bottle 102. As the IV spike 120 is rotated and further advanced into the non-ISO compliant container 102, the screw threads 135 may penetrate, “bite into,” or otherwise “dig into” the internal surface 112 of the outlet port 116. When the IV spike 120 is pulled to be withdrawn from the non-ISO compliant IV container 102, the threads 136 will secure the IV spike 120 within the non-ISO compliant IV container 102. Advantageously as a result, the tensile force required to remove or otherwise dislodge the IV spike 120 from the non-ISO compliant IV container 102 is increased and thus the IV spike 120 is better secured in the non-ISO compliant IV container 102.

Further advantageously, the penetration of the screw threads 136 into the internal surface 112 of the outlet port 116 creates a first seal between the IV spike 120 and the outlet port 116 of the non-ISO compliant IV container 102. Advantageously, the seal created prevents fluid 140 from inadvertently leaking between the IV spike 120 and the outlet port 116 of the non-ISO compliant IV container 102.

As the IV spike 120 is further advanced into the non-ISO compliant IV container 102, the screw threads 136 form a recessed path (e.g., a helical path) in the internal surface 112 of the outlet port 116. As the sealing member 135 is advanced into the outlet port 116 of the non-ISO compliant IV container 102, the sealing member 135 seals the path created by the penetration of the screw threads 136. Thus the sealing member 135 acts as a secondary seal to further prevent fluid 140 from inadvertently leaking between the IV spike 120 and the outlet port 116 of the non-ISO compliant IV container 102 in the coupled configuration.

According to various embodiments of the present disclosure, a method of manufacturing a spike 120 for an intravenous (IV) drip system 100 may include providing an elongate body 124 with a spike tip 123 at an upper portion thereof and a base 126 at a lower portion thereof. The method may further include forming a plurality of screw threads 136 protruding radially outward from an outer surface of the base 126 of the elongate body 124. In some embodiments, an outer diameter of the elongate body 124 at the base 126 is larger than that at the upper portion 127. The screw threads 124 may be formed by cutting out or removing material from the base 126 of the IV spike so as to create a series of grooves. Adjacent grooves may define the screw threads 136 therebetween, a pitch and size of which may be varied based on user's needs. In other embodiments, the screw threads 136 may be formed by adding material in the shape of the screw threads 136 to the base 126 (also referred to herein as the “lower portion”). In yet other embodiments, the screw threads 136 may be formed through other screw thread formation processes know in the art.

In accordance with some embodiments, the method of manufacturing the IV spike 120 may further include positioning a sealing member 135 between a pair of adjacent threads 136 of the plurality of screw threads 136. The sealing member 135 may be positioned so as to protrude radially outwards from the outer surface of the base 126 of the elongate body to a greater extent than the plurality of screw threads protrude radially outward from the outer surface of the base 126 of the elongate body. The sealing member 135 may be coupled, attached or otherwise bonded to the outer surface of the base 126 (or lower portion 126) of the IV spike 120 through any appropriate methods including, but not limited to ultrasonic welding, heat sealing, insert molding, gluing or other attachment methods. In other embodiments, the sealing member 135 may be coupled, attached or otherwise bonded to the outer surface of the base 126 (or lower portion 126) of the IV spike 120 through a tight interference fit. In some embodiments, the sealing member 135 may be fixedly coupled to the base 126 of the elongate body 124. In other embodiments, however the sealing member 135 may be removably coupled to the base 126 of the elongate body 124.

In accordance with some embodiments, the sealing member 135 may be formed of a flexible, resilient material which is fluid impervious. For example, the sealing member 135 may be made of a silicon material. In other embodiments, however, the valve member 35 may be formed of any non-sticking, resilient material such as natural or synthetic rubber or plastic or polytetrafluoroethylene (PTFE).

Various examples of aspects of the disclosure are described as numbered clauses (1, 2, 3, etc.) for convenience. These are provided as examples and do not limit the subject technology. Identification of the figures and reference numbers are provided below merely as examples for illustrative purposes, and the clauses are not limited by those identifications.

Clause 1: An intravenous (IV) spike for administering a medicinal fluid from a container, the IV spike comprising: an elongate body having an upper portion and a lower portion, the elongate body configured to be coupled to a drip chamber; a plurality of screw threads disposed along and protruding radially outward from an outer surface of the lower portion of the elongate body, wherein: the outer surface is configured to engage an internal surface of an outlet port of the container in a coupled configuration; and in the coupled configuration edges of the screw threads grip and engage the internal surface of the outlet port of the container to create a seal between the lower portion of the elongate body and the outlet port of the container and to retain the body in the outlet port.

Clause 2: The IV spike of Claim 1, further comprising a spike head disposed at the upper portion of the elongate body, the spike head having a puncture tip and a puncture base having a fluid inlet at an upper end thereof, wherein a fluid channel extends from the fluid inlet, through the elongate body, and into the drip chamber.

Clause 3: The IV spike of Clause 1, wherein the internal surface of the outlet port is configured without a sealing member.

Clause 4: The IV spike of Clause 3, further comprising a sealing member disposed on the lower portion and interposed between a pair of adjacent threads of the plurality of screw threads, wherein in the coupled configuration the sealing member plugs and forms a seal between the internal surface of the outlet port and the outer surface of the lower portion of the body.

Clause 5: The IV spike of Clause 4, wherein the sealing member comprises a material selected from the group consisting of rubber, polytetrafluoroethylene (PTFE), silicone, and any combination thereof.

Clause 6: The IV spike of Clause 1, wherein in the coupled configuration edges of at least one of the screw threads at least partially penetrate the internal surface of the outlet port.

Clause 7: The IV spike of Clause 1, wherein an outer diameter of each of the screw threads is larger than an internal diameter of the outlet port.

Clause 8: The IV spike of Clause 1, wherein the screw threads are formed in a series of radial bands spaced apart from each other at regular intervals along at least a portion of the lower portion of the elongate body.

Clause 9: The IV spike of Clause 1, wherein the screw threads are formed in a helical configuration at least partially along the lower portion of the elongate body.

Clause 10: An intravenous (IV) drip system comprising: an IV container containing a fluid and including a fluid outlet port having an internal surface, the internal surface being configured without a sealing member; a spike having an elongate body including an upper portion, a lower portion, and a plurality of screw threads disposed along and protruding radially outward from an outer surface of the elongate body, wherein: the outer surface of the lower portion is configured to engage the internal surface of the fluid outlet port of the IV container in a coupled configuration; and in the coupled configuration, the screw threads penetrate at least partially into the internal surface of the outlet port of the IV container to create a seal between the spike and the outlet port of the container and to retain the spike in the outlet port.

Clause 11: The IV drip system of Clause 10, wherein the spike further includes: a puncture tip and a puncture base having a fluid inlet at the upper portion of the elongate body; and a fluid channel extending from the puncture base, through the elongate body, and into the drip chamber.

Clause 12: The IV drip system of Clause 10, further comprising a sealing member disposed on the spike and interposed between a pair of adjacent threads of the plurality of screw threads, wherein in the coupled configuration the sealing member forms a seal between the internal surface of the outlet port and the outer surface of the lower portion of the elongate body.

Clause 13: The IV drip system of Clause 12, wherein the sealing member comprises a material selected from the group consisting of rubber, polytetrafluoroethylene (PTFE), silicone, and any combination thereof.

Clause 14: The IV drip system of Clause 10, wherein an outer diameter of each of the screw threads is larger than an internal diameter of the outlet port.

Clause 15: The IV drip system of Clause 10, wherein the IV container comprises a non-International Organization for Standardization (ISO) compliant IV container.

Clause 16: A method of manufacturing a spike for an intravenous (IV) drip system, the method comprising: providing an elongate body with a spike tip at an upper portion thereof and a base at a lower portion thereof; forming a plurality of screw threads protruding radially outward from an outer surface of the base of the elongate body; and positioning a sealing member between a pair of adjacent threads of the plurality of screw threads.

Clause 17: The method of Clause 16, wherein the sealing member protrudes radially outwards from the outer surface of the base of the elongate body to a greater extent than the plurality of screw threads protrude radially outward from the outer surface of the base of the elongate body.

Clause 18: The method of Clause 16, wherein positioning the sealing member comprises fixedly coupling the sealing member to the base of the elongate body.

Clause 19: The method of Clause 16, wherein positioning the sealing member comprises placing the sealing member on the base of the elongate body with an interference fit.

Clause 20: The method of Clause 16, wherein an outer diameter of the elongate body at the base is larger than that at the upper portion, and the forming a plurality of screw threads comprises removing a portion of the base material to form the screw threads.

As used herein, the terms “tubing,” “fluid line,” and any variation thereof refers to medical lines or tubes used to deliver liquids, solvents, or fluids (including gas) to or from a patient under medical care. For example, fluid lines (tubing) may be used for intravenous (IV) delivery of fluids, fluid drainage, oxygen delivery, a combination thereof, and the like.

The previous description is provided to enable any person skilled in the art to practice the various aspects described herein. While the foregoing has described what are considered to be the best mode and/or other examples, it is understood that various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the terms “a set” and “some” refer to one or more. Pronouns in the masculine (e.g., his) include the feminine and neuter gender (e.g., her and its) and vice versa. Headings and subheadings, if any, are used for convenience only and do not limit the invention.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Terms such as “top,” “bottom.” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such an embodiment may refer to one or more embodiments and vice versa.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

What is claimed is:

1. An intravenous (IV) spike for administering a medicinal fluid from a container, the IV spike comprising:
an elongate body having an upper portion and a lower portion, the elongate body configured to be coupled to a drip chamber;
a plurality of screw threads disposed along and protruding radially outward from an outer surface of the lower portion of the elongate body, wherein:
the outer surface is configured to engage an internal surface of an outlet port of the container in a coupled configuration; and
in the coupled configuration edges of the screw threads grip and engage the internal surface of the outlet port of the container to create a seal between the lower portion of the elongate body and the outlet port of the container and to retain the body in the outlet port.

2. The IV spike of claim 1, further comprising a spike head disposed at the upper portion of the elongate body, the spike head having a puncture tip and a puncture base having a fluid inlet at an upper end thereof, wherein a fluid channel extends from the fluid inlet, through the elongate body, and into the drip chamber.

3. The IV spike of claim 1, wherein the internal surface of the outlet port is configured without a sealing member.

4. The IV spike of claim 3, further comprising a sealing member disposed on the lower portion and interposed between a pair of adjacent threads of the plurality of screw threads, wherein in the coupled configuration the sealing member plugs and forms a seal between the internal surface of the outlet port and the outer surface of the lower portion of the body.

5. The IV spike of claim 4, wherein the sealing member comprises a material selected from the group consisting of rubber, polytetrafluoroethylene (PTFE), silicone, and any combination thereof.

6. The IV spike of claim 1, wherein in the coupled configuration edges of at least one of the screw threads at least partially penetrate the internal surface of the outlet port.

7. The IV spike of claim 1, wherein an outer diameter of each of the screw threads is larger than an internal diameter of the outlet port.

8. The IV spike of claim 1, wherein the screw threads are formed in a series of radial bands spaced apart from each other at regular intervals along at least a portion of the lower portion of the elongate body.

9. The IV spike of claim 1, wherein the screw threads are formed in a helical configuration at least partially along the lower portion of the elongate body.

10. An intravenous (IV) drip system comprising:
an IV container containing a fluid and including a fluid outlet port having an internal surface, the internal surface being configured without a sealing member;
a spike having an elongate body including an upper portion, a lower portion, and a plurality of screw threads disposed along and protruding radially outward from an outer surface of the elongate body, wherein:
the outer surface of the lower portion is configured to engage the internal surface of the fluid outlet port of the IV container in a coupled configuration; and
in the coupled configuration, the screw threads penetrate at least partially into the internal surface of the outlet port of the IV container to create a seal between the spike and the outlet port of the container and to retain the spike in the outlet port.

11. The IV drip system of claim 10, wherein the spike further includes:
a puncture tip and a puncture base having a fluid inlet at the upper portion of the elongate body; and
a fluid channel extending from the puncture base, through the elongate body, and into the drip chamber.

12. The IV drip system of claim 10, further comprising a sealing member disposed lower portion of the elongate body and interposed between a pair of adjacent threads of the plurality of screw threads, wherein in the coupled configuration the sealing member forms a seal between the internal surface of the outlet port and the outer surface of the lower portion of the elongate body.

13. The IV drip system of claim 12, wherein the sealing member comprises a material selected from the group consisting of rubber, polytetrafluoroethylene (PTFE), silicone, and any combination thereof.

14. The IV drip system of claim 10, wherein an outer diameter of each of the screw threads is larger than an internal diameter of the outlet port.

15. The IV drip system of claim 10, wherein the IV container comprises a non-Intentional Organization for Standardization (ISO) compliant IV container.

16. A method of manufacturing a spike for an intravenous (IV) drip system, the method comprising:
providing an elongate body with a spike tip at an upper portion thereof and a base at a lower portion thereof;
forming a plurality of screw threads protruding radially outward from an outer surface of the base of the elongate body; and
positioning a sealing member between a pair of adjacent threads of the plurality of screw threads.

17. The method of claim 16, wherein the sealing member protrudes radially outwards from the outer surface of the base of the elongate body to a greater extent than the plurality of screw threads protrude radially outward from the outer surface of the base of the elongate body.

18. The method of claim 16, wherein positioning the sealing member comprises fixedly coupling the sealing member to the base of the elongate body.

19. The method of claim 16, wherein positioning the sealing member comprises placing the sealing member on the base of the elongate body with an interference fit.

20. The method of claim 16, wherein an outer diameter of the elongate body at the base is larger than that at the upper portion, and the forming a plurality of screw threads comprises removing a portion of the base material to form the screw threads.
